(19) United States
(12) Patent Application Publication
(10) Pub. No.: US 2025/0114425 A1
(43) Pub. Date: Apr. 10, 2025

(54) Composition For Treating and Preventing COVID-19 and Method For Treatment of COVID-19

(71) Applicant: Timothy Galvin, Fayetteville, GA (US)

(72) Inventor: Timothy Galvin, Fayetteville, GA (US)

(21) Appl. No.: 18/907,432

(22) Filed: Oct. 4, 2024

Related U.S. Application Data
(60) Provisional application No. 63/542,715, filed on Oct. 5, 2023.

(51) Int. Cl. A61K 36/82; A61K 36/752; A61K 36/8962; A61K 36/9068

(52) U.S. Cl. CPC A61K 36/82; A61K 36/752; A61K 36/8962; A61K 36/9068; A61K 2236/331

(57) ABSTRACT

A composition for the treatment of or prevention of COVID-19 or long COVID may be a mixture of: water; green tea; lemon juice; red onions; and garlic. One embodiment of the composition may be made by including a plurality of bags of green tea, sliced lemons, lemon juice, cut red onion pieces, and a plurality of cut garlic pieces in water, boiling the composition for about 30 minutes, and then straining the composition. A piece of smashed ginger may be added to the composition prior to boiling. A cup of the composition may be drunk three or more times a day to treat a person having COVID-19, or a cup or more of the composition may be drunk daily to prevent COVID-19.

### BACKGROUND OF THE INVENTION

Over the last several years the world has experienced a pandemic of the disease referred to as COVID-19 which is caused by the SARS-COV-2 coronavirus and COVID-19 remains a continuing, recurring, and serious problem. Several vaccines have been developed and appear to be very effective in preventing infection or in reducing the seriousness of the disease if the virus breaks through the complete protection provided by the vaccine. However, some people because of health reasons are unable to take the vaccine and many people refuse to take the vaccine. These people are at great risk of catching the disease and are contributing greatly to the spread of the disease. Thus, there is an urgent need for treatments for the disease and methods for reducing the chance of catching the disease or reducing the severity of the disease if caught. Also, a significant number of people who catch COVID-19 have difficulty recovering from the effects of the disease and continue to suffer what is being called “Long COVID”. Symptoms of “Long COVID” may include tiredness or fatigue, difficulty thinking or concentrating sometimes referred to as “brain fog”, difficulty breathing or shortness of breath, sleep problems, and loss of smell or taste. “Long COVID” may last months or years.

### SUMMARY OF THE INVENTION

The invention provides a composition for treating the COVID-19 disease when contracted by a person and a method for treating COVID-19 by using the composition of the invention. Further, the composition of the invention has been found effective for reducing the chance of a person catching the disease and provides a method for reducing the chance of catching COVID-19. The composition is an aqueous drink comprising green tea, lemon juice, red onions, and garlic, mixed in water to form a tea drink. The composition may be referred to herein as tea. The mixture may also include ginger and the water used for making the tea may be an alkaline water. The method for treating a person who has contracted the disease is for the person to drink three cups of the tea per day, preferably at three time during the day, e.g., morning, noon, and evening for five days, which is usually enough to get rid of the symptoms and to test negative for the disease. It is also helpful for the person to drink four to six glasses of alkaline water with alkalinity of 8.8 or more per day. The alkaline water is optional as taking of the tea alone appears to be an effective treatment, but the alkaline water may increase the effectiveness of the treatment. The method for preventing catching of the disease is to drink two cups of the tea per day preferably twice during the day, e.g., morning and evening, and also drink the four to six glasses of alkaline water with alkalinity of 8.8 or more per day. Again, the alkaline water is optional as taking the tea alone appears to be an effective deterrent to catching the disease, but the alkaline water may increase the effectiveness of the deterrent effect.

### DETAILED DESCRIPTION OF THE INVENTION

A composition that has been found to be effective in treating COVID-19 comprises green tea, lemon juice, red onions, and garlic, mixed together with water to form a tea drink. Honey or sugar may be added to sweeten the tea, if desired, and to mask the onion and garlic flavors, if desired. Ginger may also be included in the composition, but is not required. Also, the tea may be made with alkaline water, but this has not been found to be necessary.

Each of the ingredients of the composition are reputed to have various health benefits. Green tea is made from unfermented leaves that are pale in color and slightly bitter in flavor. It is produced mainly in China and Japan. Green Tea is reputed to contain healthy bioactive compounds and some reports indicate that Green tea may improve brain function, may increase fat burning, may protect the brain from aging, may help to prevent type 2 diabetes, may help cardiovascular disease, may reduce bad breath, and may help a person live longer. Lemons are a yellow, oval citrus fruit with thick skin and fragrant acidic juice. Lemons are reputed to contain high amounts of vitamin C, soluble fiber, and plant compounds that give a number of health benefits, reported as aiding in weight loss, and reducing risks of heart disease, anemia, kidney stones, digestive issues, and cancer. Garlic is a strong-smelling pungent-tasting bulb, used as a flavoring in cooking and used in herbal medicine. Garlic contains a number of antioxidants and may help lower blood pressure, help lower cholesterol, may support immune function, and may reduce blood clotting. Red onions are cultivars of the onion and have a purplish-red skin and white flesh tinged with red. They are commonly used in culinary arts, but the skin of the red onion has also been used as a dye. These onions tend to be medium to large in size and have a sharp flavor and eye-watering qualities. Red onions contain sulfur compounds that are reputed to protect the body from ulcers and various cancers. They are also reputed to fight bacteria in the urinary tract. Red onions also contain quercetin which is an antioxidant which may provide protection against cancer, heart disease, and allergies. None of these ingredients are known to have any effects on COVID-19. However, the inventor has found that when these ingredients are mixed together with water in the form of a tea, the resultant tea appears to be effective in treating COVID-19 and in protecting a person against catching COVID-19.

It has been found that including ginger in the composition can be helpful. Ginger is a hot, fragrant spice made from the rhizome of a ginger plant, which may be chopped or powdered for cooking, preserved in syrup, or candied. Ginger is loaded with antioxidants and is reputed to help prevent stress and damage to a body's DNA, help fight off chronic diseases like high blood pressure, heart disease, and diseases of the lungs, and to promote healthy aging.

The amounts and proportions of the various ingredients can vary, but the following composition for the tea has been found to be effective:

10 cups of water (while not necessary, it may be beneficial to use commercially available alkaline water of 8.8 or higher alkalinity)
5 green tea bags
2 lemons sliced (add the slices of lemon between the end of the lemon to the water and squeeze the ends of the lemon into the water to get the juice out before adding the ends of the lemon to the water)
3-6 capfuls of bottled lemon juice (such as ReaLemon 100% lemon juice, the kind in the green bottle with yellow cap)
2 slices of red onion, each slice additionally sliced in half and sectioned
3 to 6 cloves of garlic, sliced
If optional ginger is used in the tea, cut about a 1 inch length of ginger root and smash the length of root
Boil the mixture for 25 to 30 minutes to make a tea.
Add honey to taste if sweetened tea is desired or allow a user to later add the honey to an individual cup of the tea, if sweetened tea is desired. Rather than using honey to sweeten the tea, sugar, preferably pure cane sugar, may be used.
Cool tea and remove the tea bags, lemon peels, etc., and strain into a storage container. The tea is then poured from the storage container into cups or glasses and heated, such as in a microwave oven, before drinking. For best results, drink the tea hot, although it can also be drunk at room temperature.

A similar composition for making a gallon of tea has also been found effective:

1 gallon of water (while not necessary, it may be beneficial to use commercially available alkaline water of 8.8 or higher alkalinity)
12 green tea bags
4-6 lemons sliced (add the slices of lemon between the end of the lemon to the water and squeeze the ends of the lemon into the water to get the juice out before adding the ends of the lemon to the water)
5-8 capfuls of bottled 100% lemon juice (such as ReaLemon 100% lemon juice, the kind in the green bottle with yellow cap)
4 slices of red onion, each slice additionally sliced in half and sectioned
5 to 8 cloves of garlic, sliced
If optional ginger is used in the tea, cut about two 1 inch lengths of ginger root and smash the length of root
Boil the mixture for 25 to 30 minutes to make a tea
Add honey to taste if sweetened tea is desired or allow a user to later add the honey to an individual cup of the tea, if sweetened tea is desired
Cool tea and remove the tea bags, lemon peels, etc. and strain into a storage container.
The tea is then poured from the storage container into cups or glasses and heated, such as in a microwave oven, before drinking. For best results, drink the tea hot, although it can also be drunk at room temperature.

While the recipes above make about ten cups of tea or about a gallon of tea, the recipes can be doubled, tripled, etc. to make larger batches of the tea. When making larger batches of tea, the amount of all ingredients are increased to maintain the approximate proportions of the ingredients as indicated in the above recipes. The exact amounts and proportions have not been found critical, but the approximate amounts and proportions should be followed. The approximate proportions will be referred to in the claims as being about the amounts of the respective ingredients listed in the above recepies. As indicated, for sweetened tea, honey may be added to taste when initially making the tea or a user can later add honey to an individual cup of the tea. Rather than using honey to sweeten the tea, sugar, preferably pure cane sugar, or other sweetener may also be used.

In the treatment of a person having COVID-19, it has been found effective for the person to drink a hot cup of the tea composition of the invention three times a day, preferably three separate times a day such as in the morning, in the afternoon, and in the evening. It has also been found effective if, in addition to the three cups of tea a day, the person also drink up to five glasses of alkaline water, 8.8 alkalinity, a day. For persons testing positive for COVID-19, following this routine for five days has resulted in such persons testing negative after the five day period of treatment. If the symptoms have not disappeared within the five days, or if the person still tests positive after the five days, continue drinking the tea three times a day until symptoms disappear and until the person tests negative for COVID-19. The daily drinking of the alkaline water is optional as taking of the tea alone appears to be an effective treatment, but the alkaline water may increase the effectiveness of the treatment. The tea has also been found effective in treating “long COVID”. The treatment of “Long COVID” would be similar to or the same as the treatment for COVID-19 indicated above. As used herein, reference to COVID-19 and to the invention as a treatment for COVID-19, COVID-19 includes “Long Covid”.

To prevent catching COVID-19, it has been found that for people exposed to others having COVID-19 (testing positive for COVID-19), drinking two cups of the tea per day preferably twice during the day, e.g., morning and evening, and also drinking the up to five glasses of alkaline water with alkalinity of 8.8 or more per day, is effective to keep the exposed person from catching COVID-19. Again, the alkaline water is optional as taking the tea alone appears to be an effective deterrent to catching the disease, but the alkaline water may increase the effectiveness of the deterrent effect.

### Applicant has Received the Following Testimonials

### Testimonial #1

I am an 80-year-old woman who has Type 2 diabetes and high blood pressure. Even though I retired as a public school teacher 17 years ago, I continue to teach piano and voice privately and presently have 30 students. With the exception of a 6-month lockdown during the height of the “shelter in place” order, I have held in-person private lessons throughout 2020 and 2021, even prior to the development of the covid vaccine. During this time, I was also drinking the tea at least 4 to 5 times a week and I strongly believe this prevented me from getting the covid virus. Unfortunately, about 8 of my students were exposed to or did get the virus once they returned to in-person school sessions, yet I remained and continue to remain virus free. I attribute it to the tea.

### Essie Fischer, Fayetteville, GA

### Testimonial #2

I believe without a doubt that this tea is the reason I am still alive today! Four days after testing positive for the Delta variant, I was rushed to the hospital with severe breathing problems and coughing fits so bad I could hardly talk. I was told that I would probably have to be placed on a ventilator which I was determined to fight. Tim had his aunt bring the tea and some alkaline water to the hospital's emergency room. Fortunately, the attendant at the front desk was familiar with the benefits of alkaline water in the fight against covid and allowed one of the nurses to bring the tea and water back to me. Four hours after drinking the hot tea, I was able to call Tim and talk without coughing, and three days later I tested negative for the covid virus. The doctors and nurses fought me constantly about the tea being in the hospital room, yet I was determined to keep it and to keep drinking it because I could feel and see the help it was providing. Unfortunately, I ran out of the tea and it took several days for someone to get out to the hospital to bring some more to me, so I continued to fight the pneumonia which had resulted from me having the virus. Once I received the second gallon of tea, I began showing significant improvement as my oxygen level started to rise. After spending 17 days in ICU, I am now home and I truly believe the tea saved my life.

### Kimberly Williams, Union City, GA

### Testimonial #3

I am one of those people who is allergic to one of the ingredients in the covid vaccine and therefore cannot be vaccinated. I also have lupus. Several months ago, both my husband and daughter tested positive to covid after being exposed to the virus by my nephew who had been in close proximity to me in the car and house. I was encouraged to make the tea, drink it myself, and give some to my husband and daughter. I believe that the tea prevented them from having severe symptoms and both recovered in a matter of days with no lingering effect. Thankfully I continue to remain virus free, and we all continue to drink the tea.

### Yessica Palmer, Lovejoy, GA

### Testimonial #4

I am a 28 year old woman who unfortunately contracted covid when I was four months pregnant. Not only was I experiencing severe symptoms, but I had also lost my sense of taste and smell. Tim, my dad, sent me the recipe and gave specific instructions on how to make the tea, how often to drink it, and to drink it along with the alkaline water. Within a few days, I had few if any symptoms and my sense of taste and smell began to return. Thankfully, my baby was born healthy and covid-free, and I have fully recovered with no negative health effects whatsoever.

### Vanessa Green, Salt Lake City, Utah

### Testimonial #5

I had COVID at the end of 2021 and became extremely ill, with persistent breathing issues, severe weakness, and crippling brain fog. It got to the point where I was considering quitting my job. In early 2022, I met Timonthy Galvin, who told me about a tea he was experimenting with to treat COVID. I was skeptical but figured I had nothing to lose, so I decided to test it for him. To my surprise, the brain fog lifted almost immediately! I also started exercising vigorously and noticed I wasn't getting winded anymore. This tea is delicious and has become an essential part of my weekly routine.

### Bethany G., Fayetteville, GA

Whereas the invention is here described with reference to specific embodiments thereof presently contemplated as the best mode of carrying out the invention in actual practice, it is to be understood that various changes may be made in adapting the invention to different embodiments without departing from the broader inventive concepts disclosed herein and comprehended by the claims that follow.

1. A composition for the treatment of or prevention of COVID-19 or long COVID comprising a mixture of:
water;
green tea;
lemon juice;
red onions; and
garlic.

2. A composition according to claim 1, wherein the water is alkaline water of 8.8 or more alkalinity.

3. A composition according to claim 1, wherein the green tea is a plurality of tea bags.

4. A composition according to claim 1, wherein the lemon juice includes fresh lemons and bottled lemon juice.

5. A composition according to claim 1, wherein the red onions are a plurality of cut red onion pieces.

6. A composition according to claim 1, wherein the garlic is a plurality of cut garlic pieces.

7. A composition according to claim 1, wherein the composition comprises about 10 cups of water, about 5 bags of green tea, slices and juice of about 2 fresh lemons, about 4 to 6 capfuls of bottled lemon juice, about 2 slices of red onions cut into pieces, and about 4 to 6 cloves of garlic sliced, and wherein the composition is boiled for about 30 minutes and then strained.

8. A composition according to claim 7, wherein the composition additionally includes ginger.

9. A composition according to claim 8, wherein the ginger is a piece of ginger root that has been smashed prior to addition to the composition prior to boiling of the composition.

10. A composition according to claim 1, wherein the composition additionally includes ginger.

11. A composition according to claim 10, wherein the ginger is about a one inch piece of ginger root that has been smashed prior to addition to the composition prior to boiling of the composition.

12. A composition according to claim 1, wherein the composition comprises about 1 gallon of water, about 12 bags of green tea, slices and juice of about 6 fresh lemons, about 6 to 8 capfuls of bottled lemon juice, about 4 slices of red onions cut into pieces, and about 6 to 8 cloves of garlic sliced, and wherein the composition is boiled for about 30 minutes and then strained.

13. A composition according to claim 12, wherein the composition additionally includes ginger.

14. A composition according to claim 13, wherein the ginger is about 2 one inch pieces of ginger root that have been smashed prior to addition to the composition prior to boiling of the composition.

15. A method for the treatment of a person with COVID-19 virus comprising the steps of:
drinking multiple cups of the composition of claim 7, wherein the composition of claim 7 is heated prior to drinking.

16. A method according to claim 15, wherein at least 3 cups are consumed per day for a period of time until symptoms of COVID-19 disappear.

17. A method for the treatment of a person with COVID-19 virus or long COVID comprising the steps of:
drinking multiple cups of the composition of claim 1, wherein the composition of claim 1 is heated prior to drinking.

18. A method according to claim 17, wherein at least 3 cups are consumed per day for a period of time until symptoms of COVID-19 disappear.

19. A method for preventing a person from getting the COVID-19 virus comprising the steps of:
drinking multiple cups of the composition of claim 7, wherein the composition of claim 7 is heated prior to drinking.

20. A method according to claim 19, wherein at least 2 cups are consumed per day.
